(19) United States
(12) Patent Application Publication
(10) Pub. No.: US 2003/0187287 A1
(43) Pub. Date: Oct. 2, 2003

(54) Synthesis intermediate products for producing vitamin d derivatives

(76) Inventors: Andreas Steinmeyer, Berlin (DE); Werner Boidol, Berlin (DE); Ludwig Zorn, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/257,767

(22) PCT Filed: Apr. 17, 2001
(86) PCT No.: PCT/EP01/04341

(30) Foreign Application Priority Data
Apr. 18, 2000 (DE) 100198619

(51) Int. Cl. C07C 401/00; C12P 33/00; C07C 69/74

(52) U.S. Cl. 552/653; 560/119; 435/52

(57) ABSTRACT

The invention relates to a new synthesis intermediate product of formula I

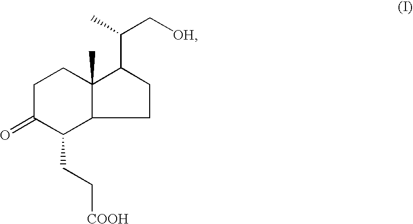

its microbiological production and use for synthesis of vitamin D derivatives.

Vitamin D derivatives have recently gained considerable importance since in addition to the known therapeutic uses (e.g., vitamin D-deficiency diseases, rickets), new types of indications are distinguished. The discovery of the “non-classic” vitamin D activities, e.g., influence of the cell growth of cancer cells, cells of the skin or cells of the immune system, points to the therapeutic potential of vitamin D derivatives in the treatment of tumor diseases, skin diseases as well as disorders of the immune system.

In nature, vitamin D derivatives occur only in extremely small amounts, so that for each pharmaceutical preparation, synthetic material must be obtained. In addition, structural variations of the active metabolite of vitamin D₃, 1α,25-dihydroxy vitamin D₃, allow the production of derivatives with altered profiles of action.

The most prominent synthesis of vitamin D derivatives with modifications in the side chain is the Barton-Hesse synthesis [D. R. Andrews, D. H. R. Barton, R. H. Hesse, M. M. Pechet, Synthesis of 25-Hydroxy Vitamin D₃ and 1α,25-Dihydroxy Vitamin D₃ from Vitamin D₂ (Calciferol). J. Org. Chem. 51: 4819-4828 (1986), EP 0010992, EP 078704, EP 078705]. The drawback of this synthesis is the use of the expensive starting material vitamin D₂, which, moreover, greatly limits the flexibility of the structural variations. Thus, for example, it is not possible to establish structural manipulations on the vitamin D skeleton in this way.

In the literature, a number of partially synthetic accesses to vitamin D derivatives are known that, while they do allow structural modifications to a greater extent, do not represent an economical raw-material base because they do not use generally accessible steroidal precursors or vitamin D₂ itself.

In addition, totally synthetic accesses exist that produce the desired target structures at high expense in a number of synthetic stages. An efficient synthesis on an industrial scale is impossible in the described cases, however.

A convergent synthesis control with a separate structural design of the CD fragment as well as the A fragment appears to be favorable in terms of an efficient but still flexible synthesis design.

While already perfected processes exist for the synthesis of the A fragment or variants thereof [e.g., E. G. Baggiolini et al. J. Org. Chem. 51: 3098-3108 (1986), Schering AG EP 766660, WARF EP 516410], the preparation of suitable CD fragments also contains big problems. If multistage total syntheses with low total yields or degradation sequences of high-grade materials, such as, e.g., vitamin D₂, are used, there is thus clearly the need to produce CD fragments from simpler precursors and at the same time to ensure a high extent of synthetic flexibility.

This invention relates to the production of the CD fragment [1R-[1α(S*),3aβ, 4β,7aα]]-octahydro-1-(2-hydroxy-1-methylethyl)-7a-methyl-5-oxo-1H-indene-4-propanoic acid I in a microbiological process as well as the product that can be produced by this process.

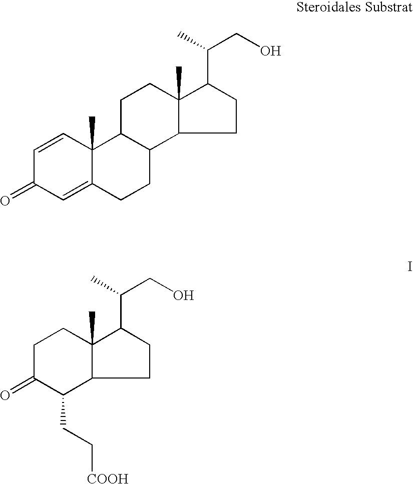

[Key:]

Steroidales Substrat=Steroidal substrate

In a screening, several microorganisms of the genera Nocardioides (Arthrobacter) and Rhodococcus (Nocardia) have been found that can implement a partial degradation of sterols and sterol derivatives. For example, the strains Nocardioides simplex (formerly Arthrobacter simplex) ATCC 13260 and Rhodococcus sp. (formerly Nocardia corallina) ATCC 13259 degrade the compound 21-hydroxy-20-methylpregna-1,4-dien-3-one (Δ1,4-HMP) [W. J. Marscheck et al. Appl. Microbiol. 23: 72-77 (1972)] to CD compound I. The use of other steroidal precursors is also possible. The latter must have a Δ-1,4-situation in the molecule and can have the commonly used steroidal-side chains in 17-position.

The degradation of 21-hydroxy-20-methylpregna-1,4-dien-3-one (Δ1,4-HMP) is preferred according to the invention.

If two formulas are referred to below with, e.g., II with radicals Z and R¹ or with IIa with radicals Z² and R², they are distinguished only to the extent that the meaning of hydrogen was added to the meaning of Z and R¹ relative to Z² and R², but all commonly used meanings are the same. This is to hold true analogously for all other formulas that are used in the application.

Compound I can be converted in a relatively simple sequence into an intermediate product that is suitable for the synthesis of vitamin D derivatives. The invention therefore also relates to a process for further processing of the compound of formula I to a compound of formula X and its further processing to a vitamin D derivative.

Thus, by esterification of the free carboxylic acid, carboxylic acid ester IIa

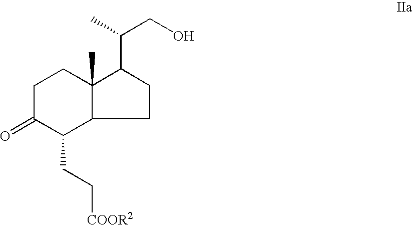

can be generated, for which it holds true that R² means a C₁-C₁₀-alkyl radical, a C₂-C₁₀-alkenyl radical, a C₂-C₁₀-alkinyl radical, whereby the alkyl, alkenyl and alkinyl radicals can be substituted by 1-5 halogen atoms, 1-5 C₁-C₅-alkoxy groups and/or can be interrupted by 1-3 oxygen atoms or 1-3 sulfur atoms, a C₁-C₅-perfluoroalkyl radical, a C₁-C₅-perchloroalkyl radical or a benzyl radical, which optionally is substituted by 1-2 halogen atoms, C₁-C₄-alkyl radicals, C₁-C₄-alkoxy radicals and/or NO₂ groups.

In principle, any ester is possible.

Preferred are compounds of formula I, in which R² means a straight-chain or branched alkyl radical with 1 to 10 carbon atoms, a trifluoromethyl radical, a trichloromethyl radical or a benzyl radical, which optionally is substituted by 1-2 halogen atoms, C₁-C₄-alkyl radicals, C₁-C₄-alkoxy radicals and/or NO₂ groups.

The C₁-C₁₀-alkyl radical can be straight-chain or branched, and/or substituted by 1-5 halogen atoms, 1-5 C₁-C₅-alkoxy groups and/or interrupted by 1-3 oxygen atoms or sulfur atoms.

The C₂-C₁₀-alkenyl radical can be straight-chain or branched, can contain 1-3 double bonds, can be substituted by 1-5 halogen atoms or 1-5 C₁-C₅-alkoxy groups and/or can be interrupted by 1-3 oxygen atoms or 1-3 sulfur atoms. C₂-C₅-alkenyl radicals with 1 to 2 halogen atoms, and 1-2 C₁-C₅-alkoxy groups, which are interrupted by an oxygen atom and/or a sulfur atom, are preferred.

The C₂-C₁₀-alkinyl radical can be straight-chain or branched, can contain 1-3 triple bonds and/or 1-3 double bonds (but at least one triple bond), and/or can be substituted by 1-5 halogen atoms or 1-5 C₁-C₅-alkoxy groups, and/or can be interrupted by 1-3 oxygen atoms or 1-3 sulfur atoms. C₂-C₁₀-alkinyl radicals with 1 to 2 halogen atoms, or 1-2 C₁-C₅-alkoxy groups, which are interrupted by an oxygen atom and/or a sulfur atom, are preferred.

Of the substituents of the benzyl radical, the methoxy group and the nitro group are preferred, especially if the latter are bonded in p-position. If, in this application, general formula II is used, radical R¹ has the same meaning as R² and in addition also the meaning of hydrogen.

The C₁-C₅-alkoxy radicals can be straight-chain or branched and can mean, e.g., methoxy, ethoxy, propoxy, or isopropoxy.

In terms according to the invention, halogen means fluorine, chlorine, bromine or iodine.

The C₁-C₅-perfluoroalkyl radical means CF₃, C₂F₅, C₃F₇, C₄F₉ or C₅F₁₁.

The C₁-C₅-perchloroalkyl radical means CCl₃, C₂Cl₅, C₃Cl₇, C₄Cl₉, or C₅Cl₁₁.

The free hydroxy group can be provided under the known conditions with a protective group, whereby the compound of general formula IIIa is obtained,

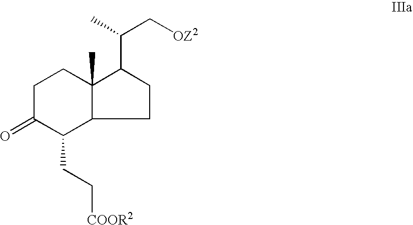

for which Z² can mean an alkyl, aryl or mixed alkyl-aryl-substituted silyl group; a (C₁-C₄-alkyl)₃Si group, a (C₆H₅)₃Si group, a (C₁-C₅-alkyl)₂(C₆H₅)Si group, a (C₁-C₅-alkyl) (C₆H₅)₂Si group, such as, e.g., the groups trimethylsilyl, di-tert-butylmethylsilyl, tert-butyldimethylsilyl, diphenyltert-butylsilyl, phenyldimethylsilyl, a tetrahydrofuranyl radical, a tetrahydropyranyl radical, a (C₁-C₅)O(C₁-C₅) group, such as, e.g., a methoxymethyl group, an ethoxymethyl group, a benzyl unit, which optionally also can be substituted with 1-2 halogen atoms, C₁-C₄-alkyl radicals and/or C₁-C₄-alkoxy radicals, a —CO—(C₁-C₆) group (e.g., an acetyl group, a pivaloyl group), a benzoyl group, whose phenyl ring optionally can have the substituents cited for the benzyl radical, or can mean another standard protective group (see T. W. Greene, P. G. M. Wuts Protective Groups in Organic Synthesis, Wiley & Sons, 1991). The silyl groups: (C₁-C₄-alkyl)₃Si group, (C₆H₅)₃Si group, (C₁-C₅-alkyl)₂(C₆H₅)Si group, (C₁-C₅-alkyl) (C₆H₅)₂Si group, as well as C₁-C₅-alkylester and (C₁-C₅)O(C₁-C₅) groups, are preferred.

If a compound of general formula III is cited in the application, Z of formula III in comparison to Z² of formula IIIa has the same meaning as Z² with the additional meaning of hydrogen, and R¹ of formula III has the same meaning as R² of formula IIIa with the additional meaning of hydrogen.

The keto group can now also be provided with a protective group, whereby the compound of general formula IVa is produced,

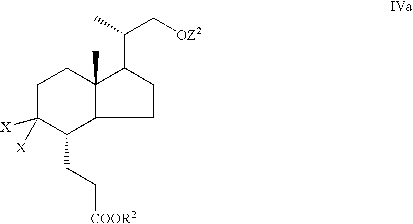

for which X, X together means a suitable protective group for a ketone, such as, e.g., a ketal of formulas —O(C₁-C₅) (open-chain), —O(C₂-C₅)O— (cyclic), especially X-OMe, OEt or X,X— —OCH₂CH₂O—, —OCH₂CH₂CH₂O— or a thioketal of formulas —S(C₁-C₅) (open-chain), —S(C₂-C₅)S— (cyclic), especially X-SMe, X-SEt, X-SPr, X-SBu, or X,X— —SCH₂CH₂S—, —SCH₂CH₂CH₂S—, which then is released again at any point of the following sequence according to known methods (T. W. Greene, P. G. M. Wuts Protective Groups in Organic Synthesis, Wiley & Sons, 1991) and optionally is used for linkage of substituents (e.g., by Wittig reactions or organometallic reactions).

As an alternative, the keto group can be removed from the molecule. This case is described by way of example of the subsequent course of synthesis. By reaction of the ketone of general formula IIIa with arylsulfonic acid hydrazides, the compound of general formula Va

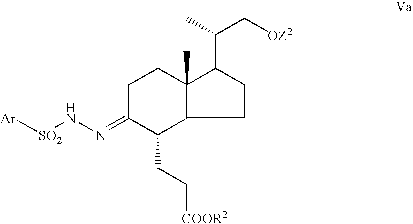

is obtained, for which it holds true that Ar is a phenyl group, which can be substituted in 1 to 3 places with a C₁-C₅-alkyl group, whereby C₁-C₅-alkyl means, e.g., methyl, ethyl, propyl, butyl, or t-butyl. The tolyl radical, the xylyl radical or the 2,4,6-trialkylphenyl radical is preferred.

By reduction of this hydrazone Va with a reducing agent (e.g., sodium borohydride, sodium triacetoxy borohydride, lithium aluminum hydride, diisobutylaluminum hydride, Birch conditions —Li or Na/liquid ammonia, etc., the compound of general formula VIa is obtained.

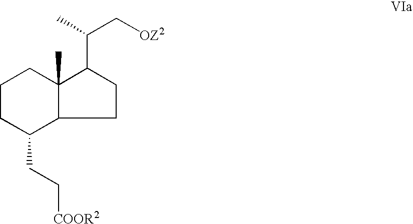

To generate a CD fragment, which can be converted into a vitamin D derivative in one of the commonly used processes, the propionic acid grouping must be degraded. For this purpose, first a double bond can be introduced in conjugation with the carboxylic acid ester unit. The reaction of derivative VIa to form the compound of general formula VIIa can be carried out by deprotonation with a base (e.g., lithium diisopropylamide, lithium diethylamide, lithium hexamethyl disilazide, sodium hexamethyl disilazide, potassium hexamethyl disilazide, sodium hydride, potassium hydride, etc.) and by subsequent quenching with phenylselenyl bromide, phenylselenyl chloride, phenylsulfuryl bromide or phenylsulfuryl chloride or comparable reagents as well as oxidation and elimination of the sulfur-containing or selenium-containing groups [e.g., H. Reich et al. J. Am. Chem. Soc. 95: 5813-5817 (1972), B. M. Trost et al. J. Am. Chem. Soc. 98: 4887-4902 (1972)]. As an alternative, other processes can also be used [e.g., Saegusa Method. I. Minami et al. Tetrahedron 42: 2971-2977 (1986)].

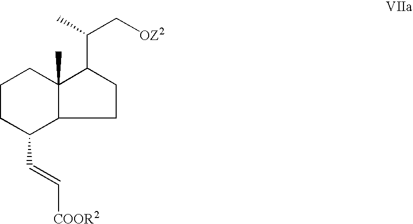

The degradation of the unsaturated ester unit can now be carried out by ozonolytic cleavage or comparable oxidative degradation processes to form the compound of general formula VIIIa.

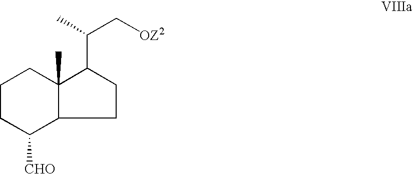

For optimal final binding of the component into known processes, the aldehyde function must now in turn be degraded. By using Baeyer-Villiger oxidation (H₂O₂-urea adduct, peracetic acid, metachloroperbenzoic acid, trifluoroperacetic acids, etc.) or related processes, the compound of general formula IXa is obtained.

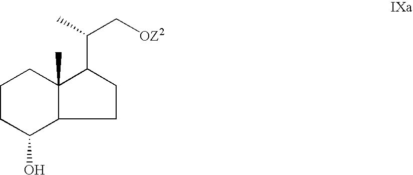

Oxidation of the hydroxy group with an oxidizing agent (e.g., pyridinium chlorochromate, pyridinium dichromate, Swem conditions, Dess-Martin conditions, etc.), the compound of general formula Xa is obtained.

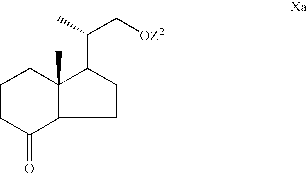

If desired, after cleavage of protective group Z², any side chain can be built up according to known processes [e.g., E. G. Baggiolini et al. J. Org. Chem. 51: 3098-3108 (1986), Schering AG: EP 421561, EP 441467, EP 450743, EP 637299, EP 639179, EP 647219, EP 649405, EP 663902, EP 832063, EP 900198].

Another subject of the invention are the intermediate stages of general formulas III and IV, as well as the intermediate stages of formulas V, VI, VII, VIII, IX and X. To finish the vitamin D system, the CD-ketone can then be reacted with a known A-ring fragment. By way of example, this is described for the compound of general formula Xa. Reaction with the A-ring-phosphine oxide XI that is known in the literature (E. G. Baggiolini et al., see above) yields the vitamin D derivative XIIa,

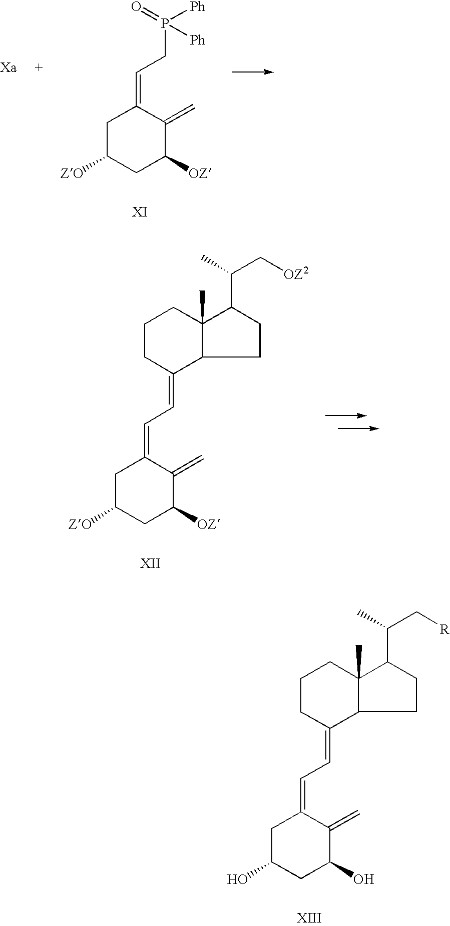

whereby for XI and XII, it holds true that Z′ can have generally the same definition as Z². To ensure compatibility, however, independent cleavage of Z² and Z′ is to be possible, e.g., if Z²=tert-butyldimethylsilyl and Z′=tert-butyldiphenylsilyl or vice versa.

Depending on the selection of the protective groups, either Z² can now be cleaved and the introduction of the side chain can be performed (see above references), or the cleavage of groups Z′ and the introduction of new protective groups Z″, which are stable under the cleavage conditions for Z², are carried out first. Moreover, other known A-fragments (e.g., XIV, XV, XVI) can also be combined with ketone Xa [K. L. Perlman et al. Tetrahedron Lett. 32: 7663-7666 (1991), S. J. Shiuey et al. J. Org. Chem. 55: 243-247 (1990), R. R. Sicinski et al. J. Med. Chem. 41: 4662-4674 (1998)].

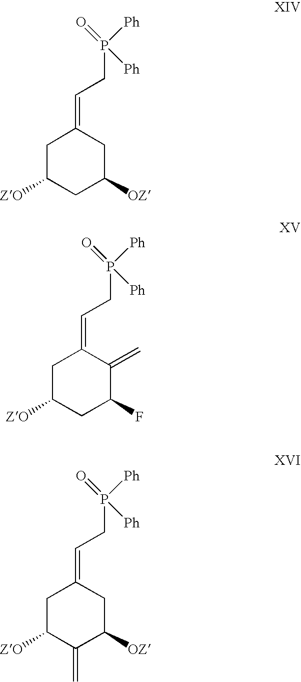

The use of ketone Xa in other synthesis accesses to the vitamin D skeleton is also possible:

e.g., after introduction of a bromovinyl unit (XVII) analogously to B. M. Trost J. Am. Chem. Soc. 114: 9836-9845 (1992) or after conversion into an enol triflate (XIX) analogously to L. Castedo Tetrahedron Lett. 29: 1203-1206 (1988), see also H. J. Knölker et al. Tetrahedron 53: 91-108 (1997).

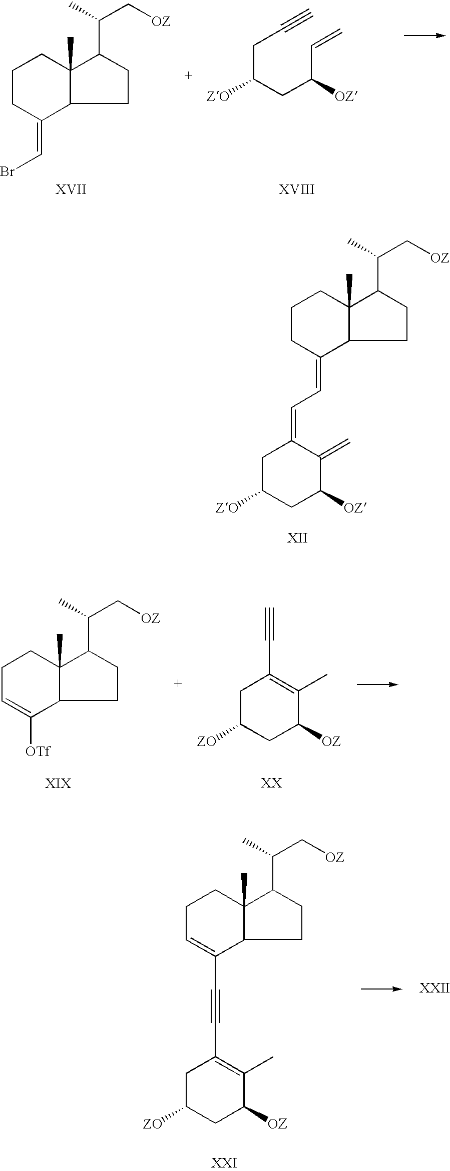

This invention contains the direct microbiological production of CD fragment I from a reasonably-priced steroidal precursor that is available in large amounts. By simple structural modifications of CD fragment I, flexible vitamin D derivatives with variations on the side chain, the A-portion and the CD-portion (use of the keto group for substitution on C-9, C-11 or C-14) can be obtained. The presence of three functional groups on CD-component I, in different oxidation stages, allows simple independent manipulations of protective group chemistry known to anyone skilled in the art. The advantageous degree of substitution of CD-fragment I, moreover, makes possible the reduction of the number of stages to produce a vitamin D derivative compared to existing processes.

In literature, several processes for producing CD fragments already exist [Henkel DE 3113053, Upjohn DE 2746323, S. Hashimoto et al. Biochem. J. 164, 715-726 (1977), U. Schömer et al. Eur. J Appl. Mikrobiol. Biotechnol. 10, 99-106 (1980), T. Nakematsu et al. Agric. Biol. Chem. 44, 1469-1474 (1980)]. None of these methods, however, allows the isolation of CD-fragments that still have side chains. The degradation ends in all cases with 17-ketone. Since specifically the synthetic structure of side chain atoms C-20 to C-22 is quite problematical in nature [P. M. Wovkulich et al. Tetrahedron 40, 2283-2296 (1984)] and since a chiral center must be generated, this process of the degradation of a steroid while retaining the chiral center at C-20 has a considerable advantage compared to the described methods.

### EXAMPLES

The examples below are used for a more detailed explanation of the subject of the invention, without intending that it be limited to these examples.

Filing

The microorganisms that are used for this invention are known and can be ordered from the following locations:

1) Nocardioides simplex, ATCC 13260

The strain was originally filed as Arthrobacter simplex under Number ATCC 13260. The actual designation reads Nocardioides simplex. It is available from the American Type Culture Collection, 10801 University Boulevard, Manassas, Va. 20110, Tel.: (703) 365-2700, Fax: (703) 365 2750 under the above-mentioned number.

2) Rhodococcus sp. ATCC 13259

The strain was originally filed as Nocardia corallina under Number ATCC 13259. The actual designation reads Rhodococcus sp. It is available from the American Type Culture Collection, 10801 University Boulevard Manassas, Va. 20110, Tel.: (703)365-2700, Fax: (703)365-2750 under the above-mentioned number.

### Example 1

### Production of the Claimed Compound I with the Strain Rhodococcus sp., ATCC 13259

Preculture

A 2 l-Erlenmeyer flask, which contains 500 ml of a nutrient solution, sterilized for 30 minutes at 120° C., consisting of 0.5% glucose, 0.5% yeast extract, 0.1% peptone and 0.2% corn steep liquor (pH 7.5), is inoculated with a slant rod culture of the strain Rhodococcus sp. (ATCC 13259) and incubated for 24 hours at 28° C. in a rotary shaker at 165 rpm.

Prefermenter

As an antifoaming agent, Synperonic, or preferably silicon SH of the company Wacker-Chemie GmbH, Munich, was used in the prefermenter and in the main fermenter.

A prefermenter that contains 10 l of sterile medium of the same composition as described for the preculture is inoculated with 500 ml of the preculture. The fermentation is carried out at 28° C., an aeration rate of 10 l/minute and an rpm of 220.

Main Fermenter

10 l of sterile medium of the same composition as in the prefermenter is inoculated with 1000 ml of the 24-hour-old prefermenter culture. The fermentation is carried out at 28° C. and an aeration rate of 10 l/minute. The rpm is varied such that the pO₂ value during the entire fermentation period is greater than 20%. An addition of the substrate Δ1,4-HMP in several stages or as a continuous addition in measured quantities has proven advantageous compared to a one-time administration. In this example, the Δ1,4-HMP was added as milling in two portions of 10 g each. The first portion is added after a growth phase of 12 hours; after the reaction is completed (about 20 hours), the second portion is added. After another 17-20 hours, the reaction is ended. The substrate is preferably used as milling, but it can also be added as a solution that is sterilized by filtration in, e.g., dimethylformamide. The control of the reaction is carried out with use of methods such as thin-layer chromatography, or preferably gas chromatography.

Harvest, Working-Up

The product is isolated from the culture broth (9.7 l) without separating the biomass. The culture broth is set at pH 8-9 with 30% NaOH solution and extracted twice with 1 volume each of methyl isobutyl ketone. The aqueous phase is set at pH 2-3 with 20% HCl solution and extracted three times with 1 volume each of methyl isobutyl ketone. The extracts are combined and evaporated to the dry state in a rotary evaporator. After the antifoaming agent that is used in the fermentation is separated, 17.15 g of crude product I, which is dissolved in acetone and filtered with silica gel, is obtained. After concentration by evaporation to the dry state, an additional crude-product batch I of 14.94 g is obtained. From this crude product, end product I can be obtained by crystallization from acetone/diisopropyl ether in a 67% yield at a purity of >98% (flash point: 98-100° C.).

Additional Reactions of CD-Fragment I

### Example 2

### [1R-[1α(S*),3aβ,4β,7aα]]-Octahydro-1-(2-hydroxy-1-methylethyl)-7a-methyl-5-oxo-1H-indene-4-propanoic acid methyl ester 1

10 g of the CD-fragment [1R-[1α(S*),3aβ,4β,7aα]]-octahydro-1-(2-hydroxy-1-methylethyl)-7a-methyl-5-oxo-1H-indene-4-propanoic acid methyl ester I is dissolved in 300 ml of benzene, 29 ml of methanol as well as a spatula tip full of p-toluenesulfonic acid are added, and the mixture is heated to boiling for 6 hours in a water separator. After cooling, it is neutralized with sodium bicarbonate solution, extracted with ethyl acetate, the organic phase is washed with sodium chloride solution and dried on sodium sulfate. The solvent is removed in a vacuum, and the residue is chromatographed on silica gel with ethyl acetate/hexane, whereby 9.8 g of title compound I is obtained as a colorless foam.

¹H-NMR (300 MHz, CDCl₃) δ 0.99 ppm (s, 3H); 1.03 (d, 3H); 3.38 (dd, 1H); 3.62 (dd, 1H); 3.63 (s, 3H)

### Example 3

### [1R-[1α(S*),3aβ,4β,7aα]]-1-[2-[[(1,1-Dimethylethyl)diphenylsilyl]oxy]-1-methylethyl]octahydro-7a-methyl-5-oxo-1H-indene-4-propanoic acid-methyl ester 2

5.6 g of ester I is dissolved in 100 ml of dimethylformamide, 2.6 g of imidazole and 5.9 ml of t-butyldiphenylsilyl chloride are added, and the mixture is stirred for 3 hours at room temperature. Then, sodium chloride solution is added, extracted with ethyl acetate, the organic phase is washed with sodium chloride solution, and dried on sodium sulfate. After concentration by evaporation, the residue is chromatographed on silica gel with ethyl acetate/hexane, whereby 8.6 g of title compound 2 accumulates as a colorless foam.

¹H-NMR (300 MHz, CDCl₃) δ 0.95 ppm (s, 3H); 1.05 (s, 9H); 1.07 (d, 3H); 3.39 (dd, 1H); 3.60 (dd, 1H); 3.65 (s, 3H); 7.38 (m, 6H); 7.68 (4H)

### Example 4

### [1R-[1α-(S*),3aβ,4β,7aα]]-1-[2-[[(1,1-Dimethylethyl)diphenylsilyl]oxy]-1-methylethyl]octahydro-7a-methyl-5-[[(4-methylphenyl)sulfonyl]hydrazono]-1H-indene-4-propanoic acid methyl ester 3

6.3 g of silyl ether 2 is dissolved in 200 ml of acetic acid and 100 ml of methanol, and 4.9 g of p-toluenesulfonyl hydrazide is added. It is stirred overnight at room temperature and then quenched with sodium chloride solution. It is extracted with ethyl acetate, washed with sodium chloride solution and dried on sodium sulfate. After concentration by evaporation, the residue is chromatographed on silica gel with ethyl acetate/hexane, whereby 5.2 g of title compound 3 accumulates as a yellowish foam.

¹H-NMR (300 MHz, CDCl₃) δ=0.73 ppm (s, 3H); 1.04 (s, 9H); 1.05 (d, 3H); 2.42 (s, 3H); 3.38 (dd, 1H); 3.58 (dd, 1H); 3.69 (s, 3H); 7.30 (d, 2H); 7.38 (m, 6H); 7.67 (4H); 7.85 (d, 2H)

### Example 5

### [1R-[1α(S*),3aβ,4β,7aα]]-1-[2-[[(1,1-Dimethylethyl)diphenylsilyl]oxy]-1-methylethyl]octahydro-7a-methyl-1H-indene-4-propanoic acid methyl ester 4

5.2 g of tosyl hydrazone 3 is dissolved in 150 ml of acetic acid, and 1.42 g of sodium borohydride is added. It is stirred for 4 hours at room temperature, and then quenched with dilute sodium hydroxide solution. It is extracted with ethyl acetate, washed with sodium chloride solution and dried on sodium sulfate. The solvent is removed, and the residue is chromatographed on silica gel with hexane/ethyl acetate, whereby 2.7 g of title compound 4 is obtained.

¹H-NMR (300 MHz, CDCl₃) δ=0.61 ppm (s, 3H); 1.03 (s, 9H); 1.06 (d, 3H); 3.35 (dd, 1H); 3.60 (dd, 1H); 3.64 (s, 3H); 7.38 (m, 6H); 7.68 (m, 4H)

### Example 6

### [1R-[1α(S*),3aβ,4β(E),7aα]]-3-[1-[2-[[(1,1-Dimethylethyl)diphenylsilyl]oxy]-1-methylethyl]octahydro-7a-methyl-1H-inden-4-yl]prop-2-enoic acid methyl ester 5

0.57 mmol of lithium diisopropylamide in tetrahydrofuran is produced, and 100 mg of ester 4 in 2 ml of tetrahydrofuran followed by 1 ml of 1,3-dimethyltetrahydro-2(1H)-pyrimidinone (DMPH) are added at −78° C. After 10 minutes at this temperature, 135 mg of phenylselenyl bromide is added in drops, and it is stirred for 90 more minutes. Then, it is quenched by adding ammonium hydrochloride solution, extracted with ethyl acetate, washed with sodium chloride solution and dried on sodium sulfate. The crude product is dissolved in 3 ml of tetrahydrofuran, cooled to 0° C., and 0.2 ml of hydrogen peroxide solution (30%) is added. After one hour at 0° C., sodium hydrogen sulfate solution is added, extracted with ethyl acetate, the organic phase is washed with sodium thiosulfate solution and sodium chloride solution and dried on sodium sulfate. The solvent is removed, and the residue is chromatographed on silica gel with hexane/ethyl acetate, whereby 78 mg of title compound 5 is obtained as a colorless foam.

¹H-NMR (300 MHz, CDCl₃) δ=0.69 ppm (s, 3H); 1.03 (s, 9H); 1.08 (d, 3H); 3.39 (dd, 1H); 3.64 (dd, 1H); 3.71 (s, 3H); 5.76 (d, 1H); 6.78 (dd, 1H); 7.37 (m, 6H); 7.68 (m, 4H)

### Example 7

### [1R-[1α(S*),3aβ,4β,7aα]]-1-[2-[[(1,1-Dimethylethyl)diphenylsilyl]oxy]-1-methylethyl]octahydro-7a-methyl-1H-indene-4-carbaldehyde 6

830 mg of ester 5 is dissolved in 10 ml of dichloromethane and 5 ml of methanol, and an ozone/oxygen mixture, which is produced in an ozone generator, is run through at −78° C. until the solution has a blue color. Then, 500 mg of triphenylphosphine is added, and the mixture is allowed to heat to room temperature. The reaction mixture is concentrated by evaporation and chromatographed on silica gel with ethyl acetate/hexane, whereby 650 mg of title compound 6 accumulates as a colorless foam.

¹H-NMR (300 MHz, CDCl₃) δ=0.69 ppm (s, 3H); 1.04 (s, 9H); 1.10 (d, 3H); 3.39 (dd, 1H); 3.61 (dd, 1H); 7.37 (m, 6H); 7.68 (m, 4H); 9.53 (d, 1H)

### Example 8

### [1R-[1α(S*),3aβ,4β,7aα]]-1-[2-[[(1,1-Dimethylethyl)diphenylsily]oxy]-1-methylethyl]octahydro-7a-methyl-1H-inden-4-ol 7

506 mg of urea-hydrogen peroxide adduct and 714 mg of potassium dihydrogen phosphate are dissolved in 8 ml of dichloromethane, and aldehyde 6 in 10 ml of dichloromethane is added in drops. Then, 0.15 ml of trifluoroacetic acid anhydride is slowly added in drops, such that the temperature of the solution does not exceed 35° C. After stirring overnight, the pH is set at 6 with 4N sodium hydroxide solution, and then sodium bicarbonate solution is added in drops until gas generation is completed. It is extracted with dichloromethane, dried on sodium sulfate and the solvent is removed in a vacuum. The residue is chromatographed on silica gel with ethyl acetate/hexane, whereby 375 mg of title compound 7 is obtained as a colorless foam.

¹H-NMR (300 MHz, CDCl₃) δ=0.66 ppm (s, 3H); 1.05 (s, 9H); 1.09. (d, 3H); 3.38 (dd, 1H); 3.54 (m, 1H); 3.61 (dd, 1H); 7.37 (m, 6H); 7.68 (m, 4H)

### Example 9

### [1R-[1α(S*),3aβ,7aα]]-1-[2-[[(1,1-Dimethylethyl)diphenylsilyl]oxy]-1-methylethyl]octahydro-7a-methyl-4H-inden-4-one 8

47 mg of alcohol 7 is dissolved in 4 ml of dichloromethane, 32 mg of pyridinium chlorochromate is added, and it is stirred for 4 hours at room temperature. Then, diethyl ether is added, and it is filtered on Celite. The solvent of the filtrate is removed, and the residue is chromatographed on silica gel with ethyl acetate/hexane, whereby 39 mg of title compound 8 is obtained.

¹H-NMR (300 MHz, CDCl₃) δ=0.61 ppm (s, 3H); 1.05 (s, 9H); 1.11 (d, 3H); 3.40 (dd, 1H); 3.61 (dd, 1H); 7.37 (m, 6H); 7.68 (m, 4H)

1. Compounds of general formula III 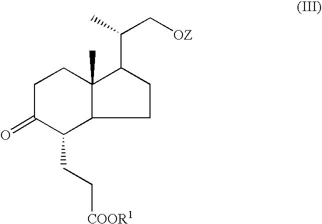
in which
R¹ means a hydrogen atom, a C₁-C₁₀-alkyl radical, a C₂-C₁₀-alkenyl radical, a C₂-C₁₀-alkinyl radical, whereby the alkyl-, alkenyl- and alkinyl radicals can be substituted by 1-5 halogen atoms, 1-5 C₁-C₅-alkoxy groups, and/or can be interrupted by 1-3 oxygen atoms or 1-3 sulfur atoms, a C₁-C₅-perfluoralkyl radical, a C₁-C₅-perchloralkyl radical or a benzyl radical, which optionally is substituted by 1-2 halogen atoms, C₁-C₄-alkyl radicals, C₁-C₄-alkoxy radicals and/or NO₂-Gruppen, and
Z means a hydrogen atom or a hydroxy protective group.

2. Compounds of general formula IV 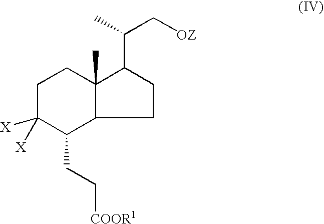
in which
R¹ means a hydrogen atom, a C₁-C₁₀-alkyl radical, a C₂-C₁₀-alkenyl radical, a C₂-C₁₀-alkinyl radical, whereby the alkyl-, alkenyl- and alkinyl radicals can be substituted by 1-5 halogen atoms, 1-5 C₁-C₅-alkoxy groups, and/or can be interrupted by 1-3 oxygen atoms or 1-3 sulfur atoms, a C₁-C₅-perfluoralkyl radical, a C₁-C₅-perchloralkyl radical or a benzyl radical, which optionally is substituted by 1-2 halogen atoms, C₁-C₄-alkyl radicals, C₁-C₄-alkoxy radicals and/or NO₂ groups,
Z means a hydrogen atom or a hydroxy protective group, and
X,X together means a keto-oxygen atom or a keto protective group.

3. Compounds of general formula IV according to claim 2 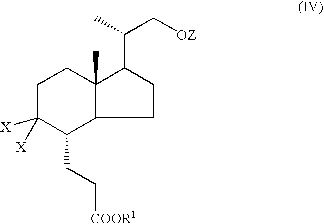
in which
R¹ means a hydrogen atom, a C₁-C₁₀-alkyl radical, a C₂-C₁₀-alkenyl radical, a C₂-C₁₀-alkinyl radical, whereby the alkyl-, alkenyl- and alkinyl radicals can be substituted by 1-5 halogen atoms, 1-5 C₁-C₅-alkoxy groups, and/or can be interrupted by 1-3 oxygen atoms or 1-3 sulfur atoms, a C₁-C₅-perfluoralkyl radical, a C₁-C₅-perchloroalkyl radical or a benzyl radical, which optionally is substituted by 1-2 halogen atoms, C₁-C₄-alkyl radicals, C₁-C₄-alkoxy radicals and/or NO₂ groups,
Z means a hydrogen atom, a (C₁-C₄-alkyl)₃Si group, a (C₆H₅)₃Si group, a (C₁-C₅-alkyl)₂(C₆H₅)Si group, a (C₁-C₅-alkyl) (C₆H₅)₂Si group, a tetrahydrofuranyl group, a tetrahydropyranyl group, a (C₁-C₅)O(C₁-C₅) group, a benzyl group that is optionally substituted by 1-2 halogen atoms, C₁-C₄-alkyl radicals, C₁-C₄-alkoxy radicals or NO₂ groups, a —CO—(C₁-C₆) group, or a benzoyl group that is optionally substituted by 1-2 halogen atoms, C₁-C₄-alkyl radicals, C₁-C₄-alkoxy radicals and/or NO₂ groups, and
X,X means an —O(C₁-C₅) group, an —S(C₁-C₅) group, together a keto-oxygen atom, a cyclic group —O(C₂-C₅)O—, or a cyclic group —S(C₂—C₅)S— or a C₆H₅—SO₂—NH—N group, whose phenyl radical is optionally substituted in 1-3 places with a C₁-C₅-alkyl radical.

4. Compound of formula I, according to claim 1

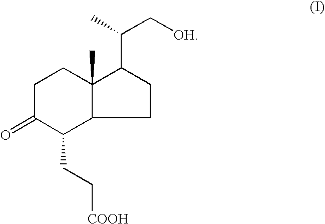

5. Process for the production of the compound of formula I according to claim 4, characterized in that the compound of formula I is obtained by a degradation reaction of a steroidal precursor with a microorganism.

6. Process according to claim 5, wherein the steroidal precursor is a steroid derivative with Δ-1,4-situation that contains any side chain in 17-position.

7. Process according to claim 6, wherein the steroidal precursor is a compound of formula

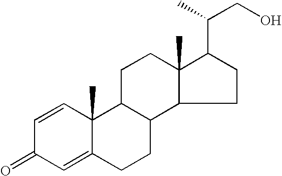

8. Process according to claim 5, wherein the microorganism belongs to one of the genera Nocardioides or Rhodococcus.

9. Process according to claim 5, wherein one of the two microorganisms is Nocardioides simplex, ATCC 13260, or Rhodococcus sp. ATCC 13259.

10. Process for further processing of the compound of general formula I 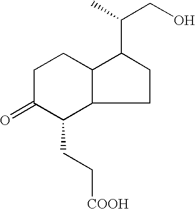
that was obtained in a microbiological stage by degradation of a steroidal precursor according to claim 5, wherein the functional groups thereof, if desired, are protected in any sequence; the keto group is optionally converted into a suitable precursor, reduced to the methylene group, and the propionic acid chain is degraded in any reaction sequence to the keto function and subsequently optionally the hydroxy protective group is removed and/or optionally other protective groups are introduced, and a compound of general formula X 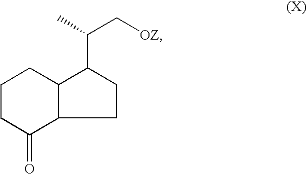
is obtained, whereby Z has the above-indicated meaning.

11. Process according to claim 10, wherein the compound of formula (X) is further processed in any reaction steps to vitamin D or vitamin D derivatives.

12. Process according to claim 10, wherein the compound of formula I is converted into an ester of general formula IIa 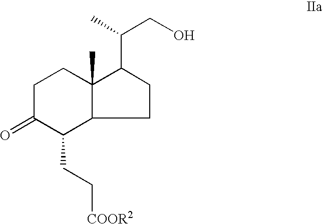
whose free hydroxy group is protected, to obtain a compound of general formula IIIa 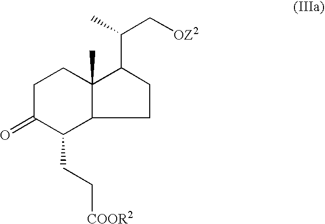
in which Z² has the meaning of Z, but does not mean hydrogen, and R² has the meaning of R¹, but does not mean hydrogen,
whose keto group is converted under common conditions into the tosylhydrazone Va 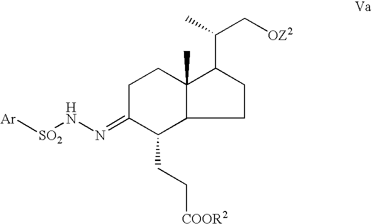
in which Ar means a phenyl group, which optionally is substituted in 1-3 places by a C₁-C₅-alkyl group, which is reduced after reduction with a reducing agent such as sodium borohydride, sodium triacetoxy borohydride, lithium aluminum hydride, diisobutyl aluminum hydride or under Birch conditions to a compound of general formula VIa 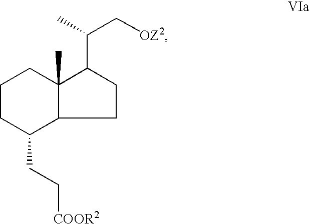
whereby if the protective groups are to have been cleaved under the conditions of reduction and their working-up, protective groups again are introduced, and their propionic acid side chain then is degraded by the reaction sequence
a) Introduction of a 2,3-double bond with lithium diisopropyl amide/DMPH/phenylselenyl bromide/hydrogen peroxide
b) Ozonolysis
c) Urea/hydrogen peroxide oxidation
d) Pyridinium chlorochromate oxidation
and is converted into ketone Xa

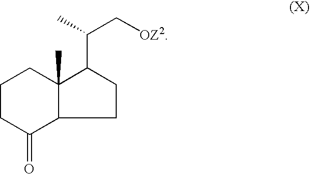

13. Intermediate products of the process according to claim 12 of general 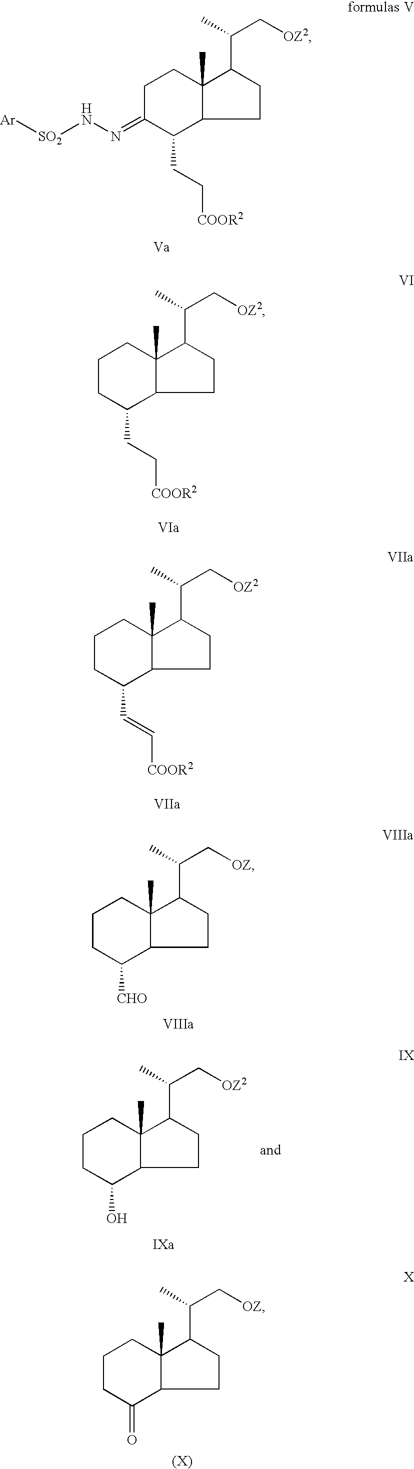
as well as their analogs, in which Z and R¹ mean hydrogen.

14. Use of the compounds of general formulas I, III, VII, and VIII for synthesis of vitamin D derivatives.

15. Steroidal degradation products that can be produced by the process according to claim 5.

16. Steroidal degradation products according to claim 14 that are produced with use of Rhodococcus sp.

17. Steroidal degradation products according to claim 14 that are produced with use of Nocardioides simplex.
